(19) United States
(12) Patent Application Publication
(10) Pub. No.: US 2005/0028815 A1
(43) Pub. Date: Feb. 10, 2005

(54) Apparatus for electronic dosage counter

(76) Inventors: Daniel M. Deaton, Apex, NC (US); Thomas W. Ruckdeschel, Apex, NC (US); Perry Genova, Chapel Hill, NC (US); Warren Jewett, Cary, NC (US)

Correspondence Address: Kos Life Sciences, Inc., 1001 Brickell Bay Drive, Miami, FL, 33131, US

(21) Appl. No.: 10/625,359

(22) Filed: Jul. 23, 2003

(51) Int. Cl. A61M 11/00

(52) U.S. Cl. 128/200.23

(57) ABSTRACT

An apparatus for dispensing medication having a canister containing medication to be dispensed to a user. The canister is movable in both a first and a second direction. The apparatus also includes a mouthpiece that provides a point of egress for the medication. The medication is dispensed to the user when the canister is moved in the first direction. Also included in the apparatus is a switch for completing an electrical circuit. The switch is activated when the canister is moved in the first direction closing the electrical circuit. The electrical circuit is opened when the canister moves in the second direction. A counter module is disclosed for performing a count upon the closure of the electrical circuit. The counter module also and displays a dispensation history of the medication in the canister. The apparatus also includes a seal for isolating the counter module from the mouthpiece and the canister.

### INTRODUCTION

1. Field of the Invention

The present invention relates to electronic monitoring and counting of medication dosages, and in particular to a metered dose inhaler that includes an electronic counter module.

2. Background of the Invention

Metered dose inhalers (“MDI”) of various configurations are known for dispensing medication into the mouth or nasal passages of a patient. Medication is expelled from the actuator and inhaled by the patient and absorbed by the mouth, nose, throat and lungs. One example is the device commonly used by asthma sufferers for dispensation of airway opening drugs. These are often called “Press & Breathe” inhalers and require simple pressing on the canister and inhalation by the user.

A pressurized metered dose inhaler (“pMDI”) is designed to deliver therapeutic agents, e.g. medicaments, to the human respiratory tract or nasal cavity. Accordingly, the MDI contains the active substance, dissolved or suspended, in a fluid propellant system that contains at least one liquefied gas in a pressurized container that is sealed with a metering valve. The actuation of the valve delivers a metered dose of medicament in the form of an aerosol spray and is directed by a suitable adapter/activator for dispensation via oral or nasal inhalation.

Another type of inhaler is the breath-activated inhaler (“BAI”). A BAI is a device typically for use with a pressurized metered dose inhaler system, and is comprised primarily of an inhalation sensing means, a means to actuate the canister automatically upon an appropriate inhalation profile, and a triggering means to communicate between the two. A BAI can be of any conventional design that has or is capable of being adapted to have, using any conventional means, such as mechanics, electro mechanics, pneumatics, fluid dynamics, a trigger force of about 0.1 to about 20 cm of water pressure. By “trigger force” is meant a force means that is minimally required by the patient to activate the dosing mechanism associated with the device. The breath-activated inhaler typically uses the suction of the user as the triggering force to release the medication.

Inhalation may be sensed by measuring changes in pressure through the device or by measuring flow rate, directly or indirectly and separately or in combination. The literature is replete with methods for accomplishing this and includes moving vanes or flaps, elastomeric diaphragms, electronic pressure sensors, flow sensors, and combinations of mechanical sensors with electronic timing circuits.

The canister may be actuated by mechanical (e.g. springs, levers, etc.) electromechanical (e.g. solenoids, motors) or pneumatic means. The canister may be actuated and remain in the actuated position until intervened upon by the patient or may be caused to dwell in the actuated position for some duration returning automatically to rest position without any intervention.

Traditional inhaler devices are known to be confusing to the user with respect to the number of doses remaining in the canister at any one time. Accordingly, the user is faced with the possibility of running out of necessary medication at a critical time. Alternatively, the user must carry additional costly medication at all times to insure that it is always on hand. Further, the disposal of a canister of medication when there are still a number of doses remaining can lead to increased expense in the treatment of an ailment.

Still further complications with the traditional inhalers mean that a user is forced to manually determine the timing between dosing. As a result it is up to the user to insure that a proper time period has expired between dosing to prevent an overdosing of medication. Similarly, many medications have a maximum threshold for dosing over a specific period. As a result overdosing can occur when more than the predetermined number of doses are administered in a set period, for example 24 hours. Once again it is up to the user to ensure that no more than the maximum number of doses is taken over the time period. In addition, the medications may require a sequence of multiple device activations to deliver a complete dose. The user must accurately monitor these activations. With the state of current medical treatments, often a user will have multiple drugs prescribed for the treatment of a single malady. When coupled with the irregularity of the dosing schedules improper dosing of a patient becomes a genuine concern.

Accordingly, the present invention is directed to an apparatus that overcomes the problems associated with traditional inhalers. The present invention is related to an inhaler that provides information to the user regarding the dosage administration.

These and other characteristics of the present invention will become apparent from the further disclosure to be made in the detailed description given below.

### SUMMARY OF THE INVENTION

In accordance with the present invention an apparatus related to the dispensation of medication is disclosed. The apparatus includes a canister containing medication to be dispensed to a user. The canister is movable in both a first and a second direction. The apparatus also includes a mouthpiece that provides a point of egress for the medication. The medication is dispensed to the user when the canister is moved in the first direction. Also included in the apparatus is a switch for completing an electrical circuit. The switch is activated when the canister is moved in the first direction closing the electrical circuit. The electrical circuit is opened when the canister moves in the second direction. A counter module is disclosed for performing a count upon the closure of the electrical circuit. The counter module also displays a dispensation history of the medication in the canister. The apparatus also includes a seal for isolating the counter module from the mouthpiece and the canister. This isolation assists in the prevention of contamination of the counter module. Additionally, any gaseous or particulate emissions from the counter module are isolated from the inhalation airflow path.

The dispensation history can include, but is not limited to, the number of doses of medication remaining in the canister, the number of doses taken of a dosage sequence, number of doses taken over a period of time, and time since the last dispensation of the medication.

The switch may be an electrically conductive contact imbedded in the seal. Alternatively, the switch may be formed on a circuit board and be acted upon by a protrusion in the seal as the canister is moved in the first direction. The switch may also be acted upon directly by a ferrule portion of the canister, the switch being isolated from the canister by a second seal. Other arrangements of the switch include the entire seal being made of conductive material to close contacts on the circuit board.

In one embodiment the switch is a water resistant dome switch. The dome switch may be mounted in a variety of locations including substantially parallel to an axis of travel of the canister and acted upon by a ferrule of the canister. Other arrangements of the dome switch include on a platform that extends perpendicular to the axis of travel of the canister into the mouthpiece and acted upon by an end portion of the canister. Another arrangement of the dome switch is on a top surface of an actuator sump and acted upon by an end portion of the canister. Still another arrangement of the dome switch is in an actuator sump and acted upon by the actuator as the canister is depressed. Alternatively, the dome switch may be located on an exterior surface of the mouthpiece and depressed when the user depresses the canister against the mouthpiece.

Another switch that might be used includes at least two open contacts that are in electrical communication with the counter module, and utilizes a conductive surface of the canister to close the circuit. In one arrangement the open contacts are located on a top surface of an actuator sump and are acted upon by a metallic end portion of the canister.

In another embodiment the switch may be formed of a movement sensor such as a light sensor, acoustic sensor, a Hall effect or magnetism sensor, or a pressure sensor.

The light sensor emits light and receives a reflected signal. Upon movement of the canister the reflected signal is altered. This alteration is detected by the sensor and provides a change of position signal to circuitry, initiating a count.

The acoustic sensor emits an acoustic signal and receives a reflected signal, upon movement of the canister the reflected signal is altered and the sensor detects this alteration. Alternately, the acoustic sensor emits no signal, but receives and recognizes the acoustic “signature” of the aerosolization of the metered dose.

The magnetic sensor senses the movement of the canister by detecting changes in the magnetic signature of the canister or a fero-magnetic element attached thereto as it is moved in the first and second directions.

The pressure sensor may be arranged in an actuator sump and detects a change in pressure upon the dispensation of the medication from the canister.

Further characteristics, features, and advantages of the present invention will be apparent upon consideration of the following detailed description of the invention taken in conjunction with the following drawings, and in which:

### BRIEF DESCRIPTION OF THE DRAWINGS

FIG. 1 is a side view of an inhaler according to one embodiment of the present invention;

FIG. 2 is a perspective view of an inhaler according one embodiment of the present invention;

FIG. 3 is a rear view of an inhaler of according to one embodiment of the present invention;

FIG. 4 is an internal arrangement view of an inhaler according to one embodiment of the present invention;

FIG. 5 is a perspective cross-sectional view of an inhaler according to one embodiment of the present invention;

FIG. 6 is a profile cross-sectional view of an inhaler according to one embodiment of the present invention;

FIG. 7 is a block diagram of a counter module according to one embodiment of the present invention;

FIG. 8 is an inhaler according to one embodiment of the present invention having the counter module mounted on the mouthpiece side of the apparatus;

FIG. 9 is an inhaler according to at least one embodiment of the present invention having a circuit board mounted switch or alternatively having conducting members located in the membrane impinge upon open contacts on the circuit board;

FIG. 10 shows an inhaler according to one embodiment of the present invention with the entire membrane/seal made of conductive elastomer to contact with exposed contacts on the circuit board;

FIG. 11 shows an inhaler according to one embodiment of the present invention having a mechanical switch mounted directly to the circuit board extending into the mouthpiece to contact canister motion directly;

FIG. 12 shows an inhaler according to one embodiment of the present invention having a water resistant dome switch mounted on a flexible circuit in the path of canister to sense motion of the canister;

FIG. 13 shows an inhaler according to one aspect of the present invention having a water resistant dome switch mounted on a platform in the path of canister to sense motion of the canister bottom;

FIG. 14 shows an inhaler according to one embodiment of the present invention wherein the bottom of the ferrule completes the circuit when it touches exposed contacts;

FIG. 15 shows an inhaler according to one embodiment of the present invention having dome switches wherein the bottom of the ferrule completes the circuit when it compress the dome switch;

FIG. 16 shows an inhaler according to one embodiment of the present invention having board-mounted sensors to detect the motion of the canister including optionally optical, acoustic, or Hall effect sensors;

FIG. 17 shows an inhaler according to one embodiment of the present invention having circuit components mounted on flexible circuitry around the bottom of the mouthpiece;

FIG. 18 shows an inhaler according to one embodiment of the present invention having a dome switch is mounted directly in line with the actuator;

FIG. 19 shows an inhaler according to one embodiment of the present invention having a dome switch mounted externally on the mouthpiece;

FIG. 20 shows an inhaler according to one embodiment of the present invention having a pressure sensor to detect pressure directly in the actuator sump region;

FIG. 21 shows a graphical representation of an actuation of an inhaler according to one embodiment of the present invention;

FIGS. 22a and 22b show an inhaler according to one embodiment of the present invention before and during closing of a switch with an elastomeric seal and ramp

FIGS. 23a and 23b show ramp profiles according to at least one embodiment of the present invention for a membrane switch and a contact switch respectively; and

FIG. 24 shows two inhalers having differing nozzle configurations according to the present invention.

FIG. 25 shows a multi-component inhaler housing according to one embodiment of the present invention.

### DETAILED DESCRIPTION

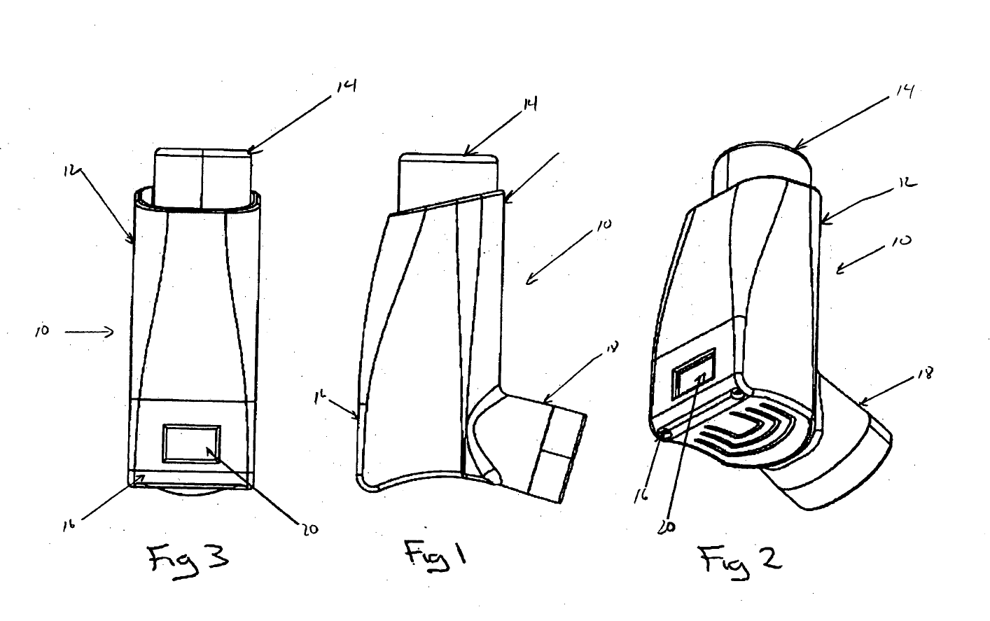

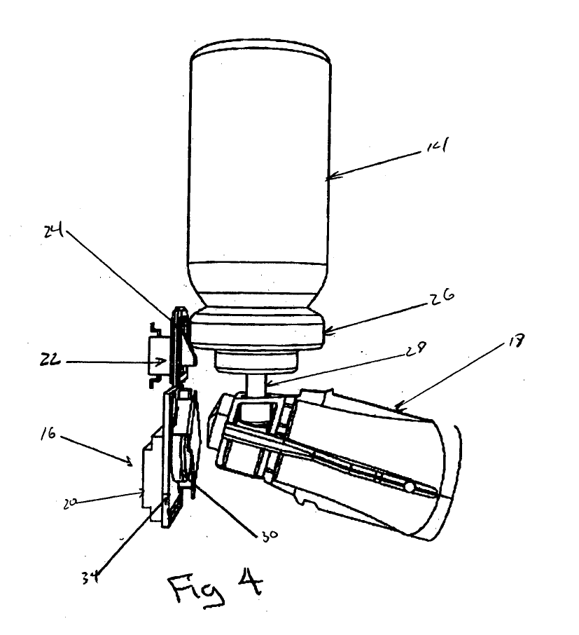

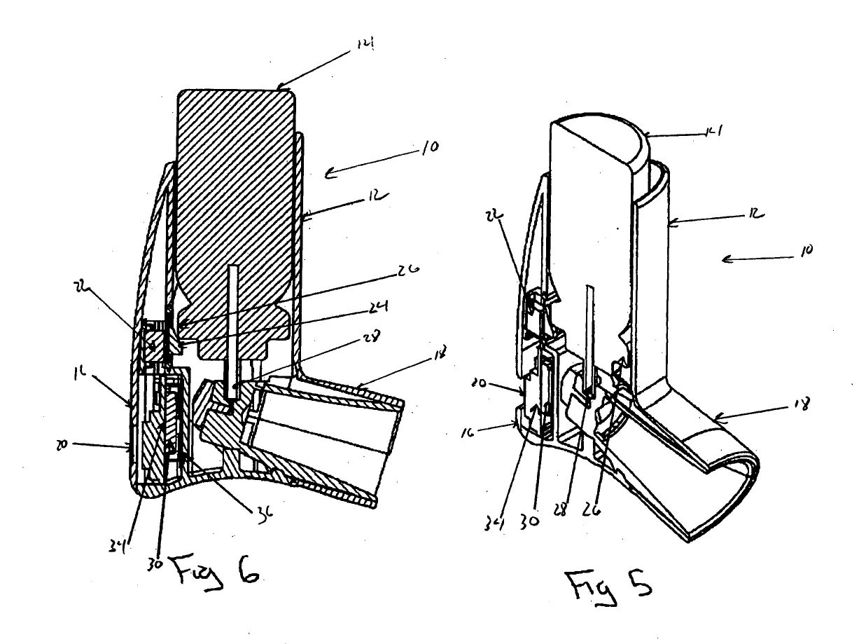

An inhaler 10 in accordance with one aspect of the present invention is shown in FIG. 1. The inhaler is comprised of a canister holder 12, a canister of medication 14, a counter module 16, and a mouthpiece 18. The counter module 16 includes a display 20, and will be discussed in detail below. FIG. 2 shows a perspective view, and FIG. 3 shows a rear view of the inhaler 10.

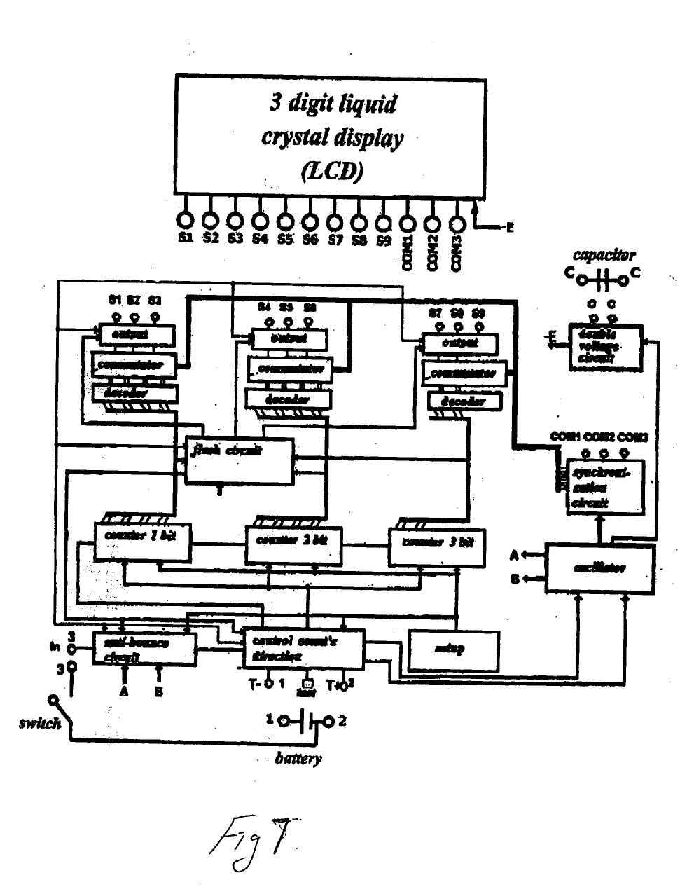

FIG. 4 depicts an internal view of an inhaler 10 according to one aspect of the present invention. The canister 14 contains the medication that is to be administered to the patient. The canister 14 includes an actuator 28 that releases the pressurized medication when it is depressed in the direction of the canister 14. Sealing one end of the canister 14 is a ferrule 26.

Also shown in FIG. 4 is the counter module 16, and the display 20 electrically connected thereto. In FIG. 4 the display is shown physically mounted to the counter module 16, however, other arrangements of the two components may be made without departing from the scope of the present invention. A battery 30 provides the power necessary to operate the counter module 16 including the display 20. Also provided as part of the counter module 16 is a switch 22. As shown in FIG. 4, the switch is mounted external to a printed circuit board 34 and isolated from the canister 14 and mouthpiece 18 by an elastomeric switch seal 24. The switch 22 is electrically connected to circuit board 34, using wires or flexible circuitry (not shown).

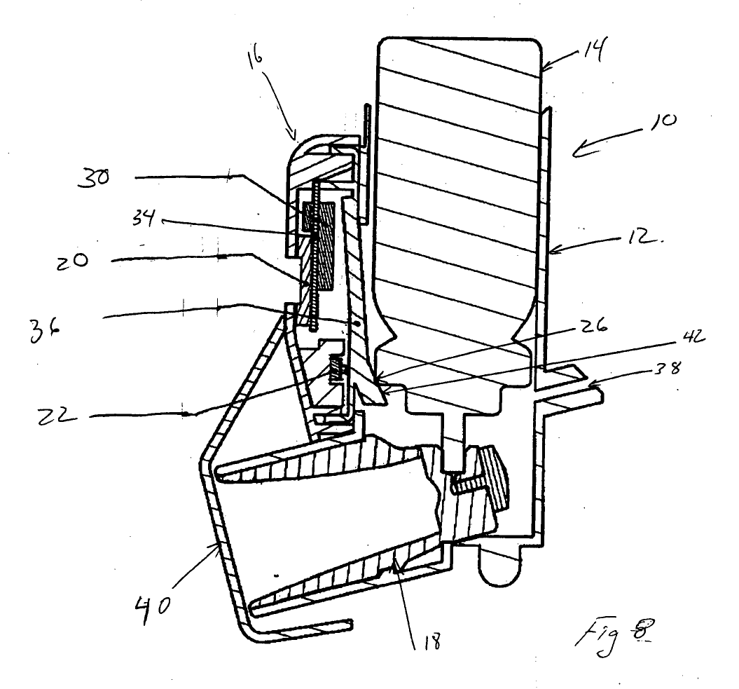

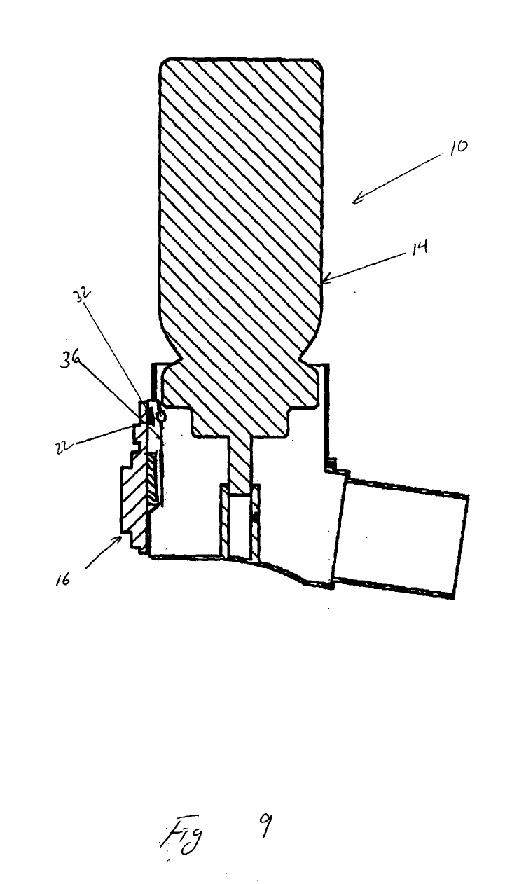

FIGS. 5 and 6 depict the components shown in FIG. 4 as a cross-section incorporated into a complete inhaler. The prevention of cross contamination between the counter module 16 and the airflow pathway is desirable. Accordingly, in FIG. 6, a seal wall 36 is shown isolating the components of the counter module 16 mounted on circuit board 34 from the remainder of the inhaler 10. Similarly, the switch seal 24 isolates the switch 22 from the inhaler 10. The isolation of the counter module 16 from the mouthpiece 18, the canister 14, and the canister holder 12 allows the user to remove the canister 14 and wash the apparatus without fear of damaging the components of the counter module 16. The use of the seals 24 and 36 make the counter module substantially water resistant. Additionally, the seals 24 and 36, prevent contaminants from the counter electronic componentry from entering the inhalation airflow path.

The counter module 16 is comprised of a circuit board 34 for mounting all or substantially all of the components of the counter module 16. These components include the battery 30, the display 20, the switch 22, and an application specific integrated circuit (ASIC). The counter module 16 can operate in a variety of counting modes. The manufacturer may select the mode of the apparatus during production. Alternatively, the user may select the mode in an apparatus that is enabled with two or more counting modes. Examples of the modes are discussed in detail below.

The various modes of operation of the counter include at least the following examples. In one example the counter operates in a single function mode, the doses remaining mode. In this mode the counter is designed to decrement from a predetermined starting number each time the switch 22 is activated. In one application, the display 20 may be an LCD having three digits and be large enough to be readable at arm's length in ordinary light at an angle of 30°. The leading zero of the display is typically blanked unless the canister comprises greater than 99 doses of medication. In a further application, the display will read normally displaying the number of doses remaining in the canister until only a set number remain, for example 20 doses. Upon reaching the 20 dose remaining point, the display flashes once per second to indicate to the user that the canister is nearing the empty point. When the count reaches 0, one of the LCD digits will flash zero to indicate that the canister is empty. This flashing signals the user that the canister 14 is empty.

In a second example, the counter operates in a dual function mode, the doses remaining plus dosing sequence mode. In this embodiment the doses remaining portion of the counter operates as described above. In addition, the display 20 will indicate the number of doses taken within a dosing sequence, for example actuation 1 of a total of 3. This mode may be indicated by a segment from the leading digit, a legend, or a symbol may identify the function being shown by the display 20. In one application, the number of doses taken in the sequence is displayed immediately after a dosing of the medication. The mode may automatically switch back to the doses remaining mode after a pre-set period of time, for example, two minutes. Alternatively, the user may toggle between the two modes as desired.

In a third example, the counter operates in a three function mode, time elapsed since last dose, number of doses in last twenty-four (24) hours, and doses remaining. The doses remaining function operates as described above. The time since the last dosing function includes a time displayed in hours and tenths of hours and any zeros to the left of the indicated time are blanked. This time represents the time since the last depression of the canister 14 in the apparatus. Upon depression of the canister 14 a timer is started. This timer continues running and is reset to zero upon a subsequent depressing of the canister 14. At this subsequent depression of the canister 14 the timer again counts the time period till the next depression of the canister 14. In this fashion a running time between doses is systematically updated. The third mode indicates the number of doses delivered in the preceding twenty-four hour period. Upon an initial depression of the canister 14, a continual clock twenty-four hour is started. The clock registers the number of times during the twenty-four hour period the canister has been depressed. In this fashion the number of doses delivered over that time period is registered by the device and displayed for the user. Alternatively, the user may toggle between the multiple display modes of the apparatus.

Along with selecting the mode that an apparatus will operate under the manufacturer may select the initial number from which the apparatus will decrement 1 with each operation of the apparatus. This will be accommodated by the use of the ASIC which is programmable and provides for mode selection by the manufacturer. Typical dosage containers provide for example 60, 100, 120, 150, 200, and 400 doses of the medication. It is understood that other dosages could be used without departing from the scope of the present invention.

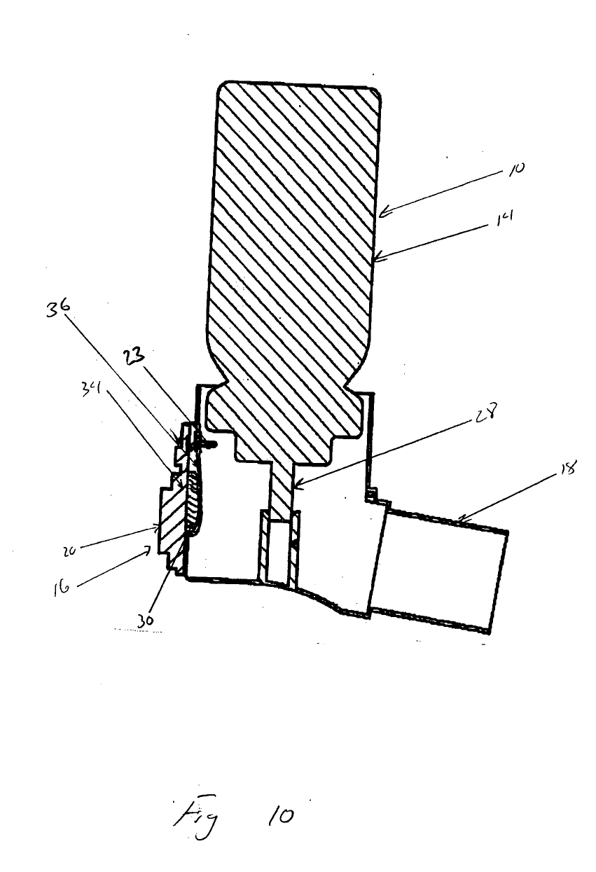

FIG. 7 is an example of a block diagram of the circuitry of the counter module 16 including, the switch 22, the battery 30 and the display 20. Among the features of the circuit shown in FIG. 7 is a setup feature. This enables the manufacturer to establish the initial dose count from which each actuation of the apparatus will be reduced. Similarly, there is a control for the direction of the count. In certain applications it may be preferable for the count to increment rather than decrement. This alteration to the device can be made by the manufacture through the connection of the count direction terminals. The switch 22 is also depicted on the block diagram. The switch 22 provides the input data that is processed by the circuit to produce the displays on the display 20, as shown in FIG. 7 a LCD.

Also among the features of the circuit is an anti-bounce circuit. The anti-bounce feature prevents the counting of erroneous signals by ignoring the depression of the switch that last less than 50 ms. As a result the shock associated with a fall of the apparatus will not register a count because it will not close the electrical switch for a sufficient period of time. Further, to prevent double counting of dosing, the apparatus will not permit a count less that 500 ms±75 ms after a preceding count. Still further, should the device receive a shock that interrupts the power supply from the battery, the anti-bounce features retains the count over a short duration, such as 100 ms. Upon restoration of normal power, the count is restored to the value before the power interruption.

Other features of the circuit include a double voltage circuit that takes the battery produced 1.5 vdc and produces 3 vdc. In certain applications three volts are necessary to drive the display, in particular a LCD. Oscillator circuitry is utilized to generate the anti-bounce interval as discussed above. Similarly, the flash rate of the display is also controlled by the oscillator circuitry. For example, when the doses remaining fall below 20, the display flashes at a certain interval to alert the user of the low dosage number. The interval for the flash, once per second, or once per half-second is set by the flash rate and controlled by the oscillator circuitry.

Another function of the oscillator is to set the display drive frequency. LCD's, for example are typically configured to conserve power. At certain frequencies, the human eye cannot detect that a light is not continuous. Accordingly, to conserve power the LCD is not continuously illuminated, but rather is illuminated at a certain cycle rate. This rate is at sufficient speed as to look to the human eye as if it were continuous. Reducing the amount of time that the LCD is actually illuminated reduces the energy consumption of the apparatus accordingly this cycle rate us established by the oscillator circuitry. Other elements shown in FIG. 7 including counters, decoders, and commutators are components necessary for driving the display and are well known to those skilled in the relevant arts.

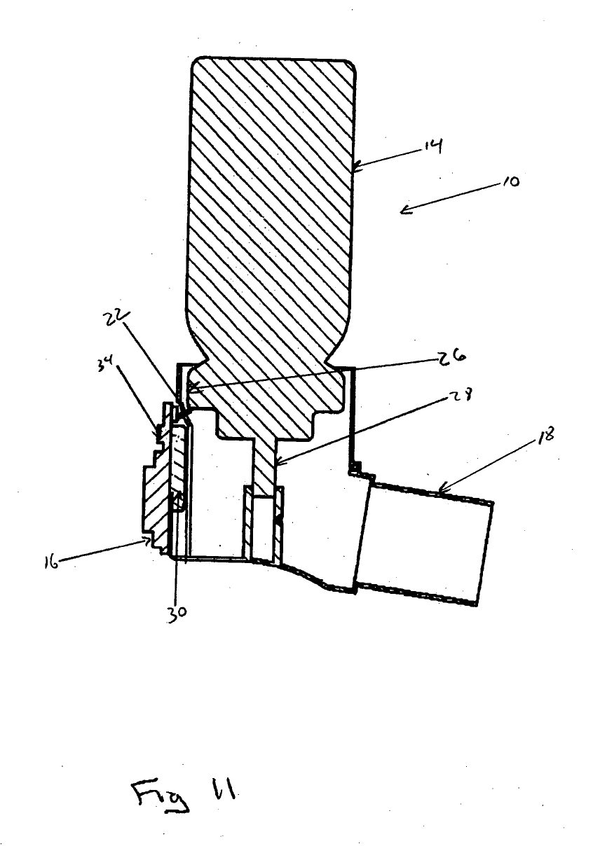

FIG. 8 shows an alternative configuration of the present invention. As shown in FIG. 8, the counter module is located on the mouthpiece side of the apparatus. Such orientation may be beneficial for the user as the display 20 is located on the same side as the mouthpiece and doe not require the user to turn the device over to view. Further, such a configuration may be necessary in instances where an increased airflow through the mouthpiece is desired. As can be seen in FIG. 8 a vent 38 is located on the backside of the apparatus. The necessity of such a feature forces the display module 16 to be moved to a more convenient location. Another feature of the apparatus shown in FIG. 8 is the cover 40 that prevents debris to enter the mouthpiece when not in use.

In the apparatus shown in FIG. 8, a single elasotmeric seal 36 is used to isolate the entire display module 16 from the remainder of the inhaler 10. The elastomeric seal 36 includes a protrusion or ramp 42 and is acted upon by the ferrule 26 of the canister when the canister is depressed in the direction of the mouthpiece. The ramp 42 is forced away by the ferrule 26 and closes the contacts on switch 22 to activate the counter module 16.

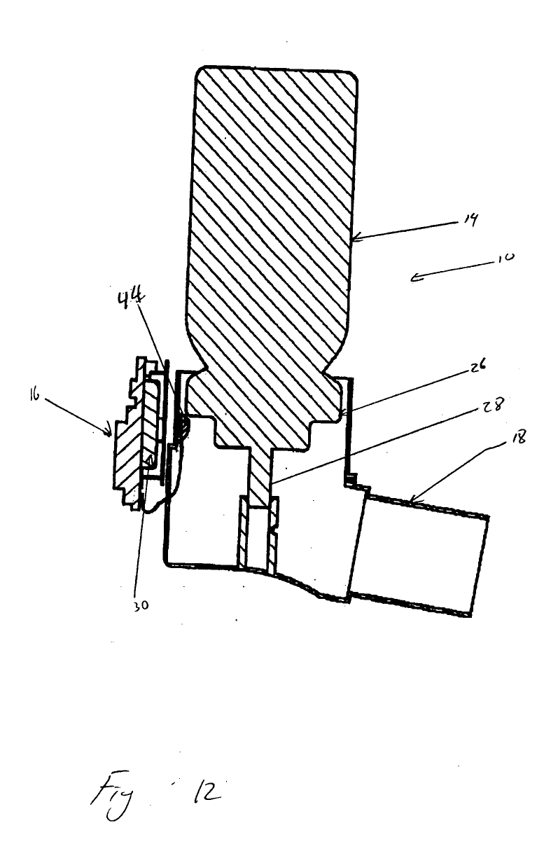

Similarly FIG. 9 shows an inhaler 10 where all of the counter components are sealed from the air pathway via a flexible seal 36. The seal 36 deforms as the canister 14 moves and communicates with the circuit board 34. In one embodiment the switch component 22 is mounted directly to the board 34. Alternatively, the seal 36 may include a conductive portion 23 that closes contacts on the circuit board 34, when depressed by the movement of the ferrule 26, as shown in FIG. 10.

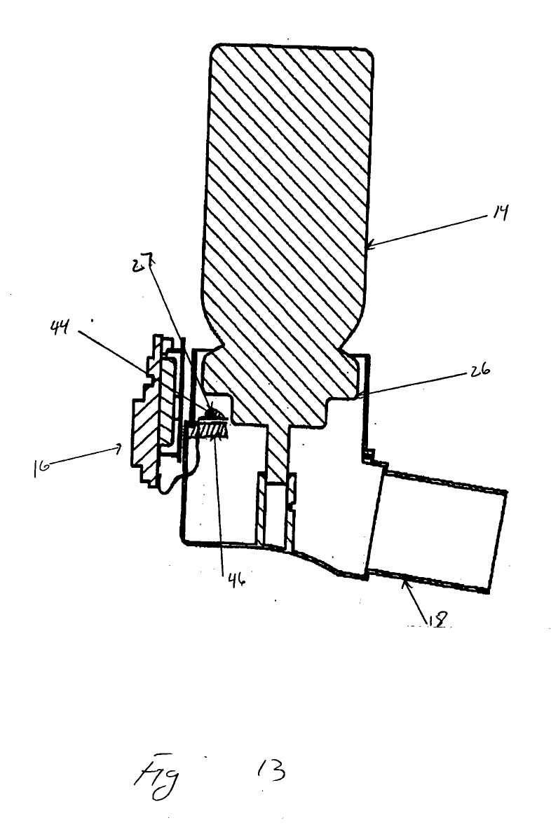

In FIG. 11 a mechanical switch mounted directly to the circuit board 34 is shown. The switch 22 extends into the mouthpiece 18 contacting the canister 14 at ferrule 26. Upon depression of the canister 14, the switch 22 is triggered as the ferrule 26 passes.

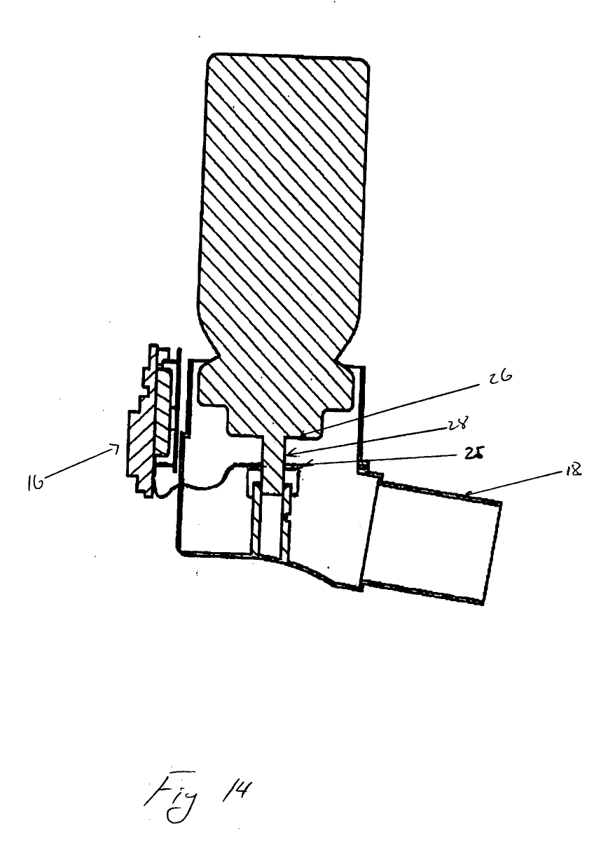

FIG. 12 depicts a water resistant dome or membrane switch 44 mounted in the path of ferrule 26 of canister 14. The dome switch 44 senses motion of the canister 14 as ferrule 26 passes the switch thereby closing contacts of the switch 44. The dome switch 44 is electrically connected to the counter module 16.

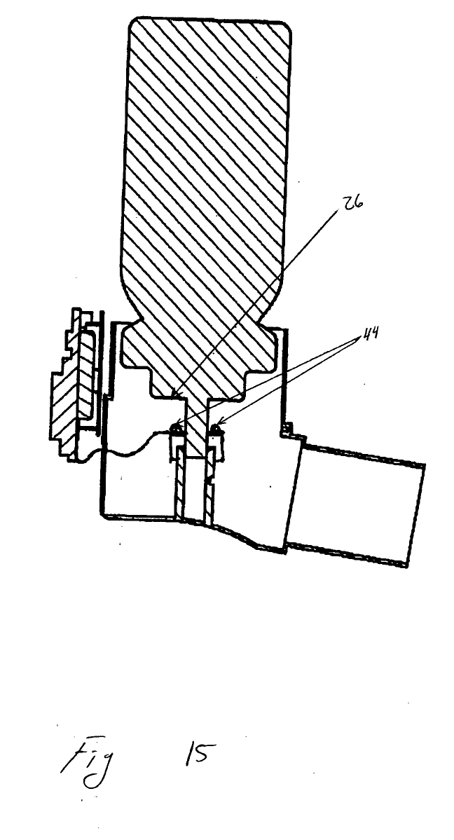

FIG. 13 shows a dome switch 44 mounted on a platform 46 in the path of canister 14. As the canister 14 approaches the bottom of its travel contact is made between the dome switch 44 and the ferrule 26 of canister 14. In this case, allowances for the over travel of the canister are very important, as it is necessary to insure that the canister 14 closes the dome switch 44 at or near the end of its travel in the direction of the mouthpiece 18. If the dome switch 44 is located too close to the canister 14 then it will impede the travel of the canister 14 and prevent full discharge of the medication. Alternatively, if the dome switch 44 is located too far from the canister 14 then the contacts of the dome switch 44 might not be closed during dispensation of the medication to the user thus defeating the usefulness of the counter module 16.

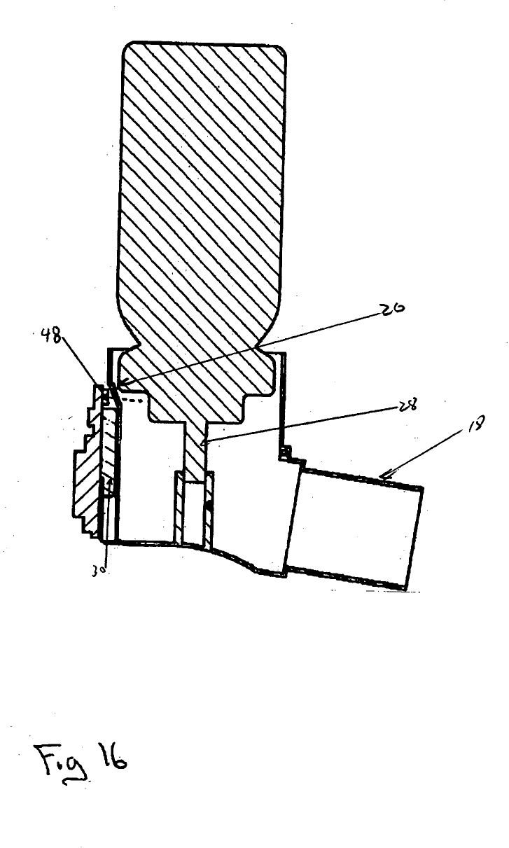

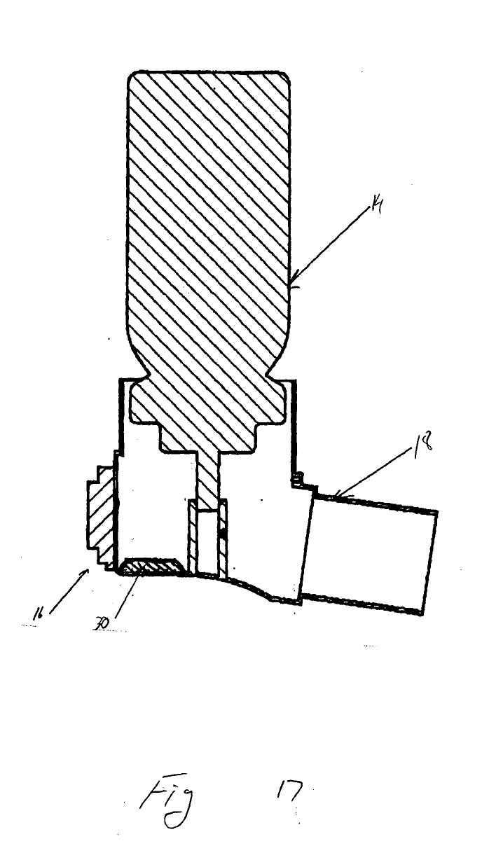

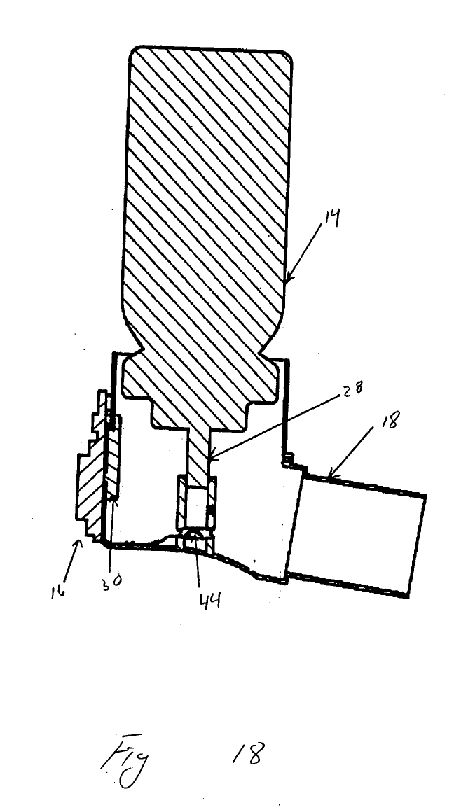

In FIG. 14 flexible circuit with exposed contacts points 25 is shown that is disposed around the actuator 28. Typically, the canister 14 is made of a metallic or conductive material such as aluminum or steel. As a result, the contacts 25 are open until the canister is depressed to dispense the medication. Upon reaching the bottom of travel the ferrule 26 of the canister 14 touches the contacts 25. As the ferrule 26 is made of a conductive material the canister 14 completes the circuit when it touches the exposed contacts 25. Coaxial alignment of the contacts 25 and actuator 28 help to ensure a highly repeatable contact point. Here as was the case with the apparatus shown in FIG. 13 allowances for the over travel of the canister are important. However, use of an elastomeric cushion 27 addresses the travel distance issue without requiring tight tolerances. The contacts 25 are electrically connected to the counter module 16. FIG. 15 shows a similar configuration to that of FIG. 16 except instead of using the conductive nature of the ferrule material to complete the circuit, dome switches 44 are used to complete the circuit when the ferrule 26 puts pressure on them sufficient to close the internal contacts.

In FIG. 16 an inhaler is shown that does not use traditional contacts to close the electrical circuit. Instead a sensor 48 is used to determine whether the canister 14 has moved. Among the types of sensors that may be used are optical sensors, acoustic sensors, and Hall effect or magnetic sensors. When any of these sensors detects movement of the canister, that movement is communicated to the counter module 16 and is registered on the display 20. The optical sensor emits light that reflects from metal ferrule and is detected by a chip in the sensor. The acoustic sensor transmits and receives an acoustic signal to sense distance to the ferrule 26, this distance can be used to detect motion. Alternately, the acoustic sensor does not transmit, but receives and recognizes the acoustic “signature” of the aerosolization of the metered dose. The Hall effect sensor detects a change in the magnetic field around the sensor caused by the motion of the metallic canister 14. Other sensors could also be used without departing from the scope of the present invention.

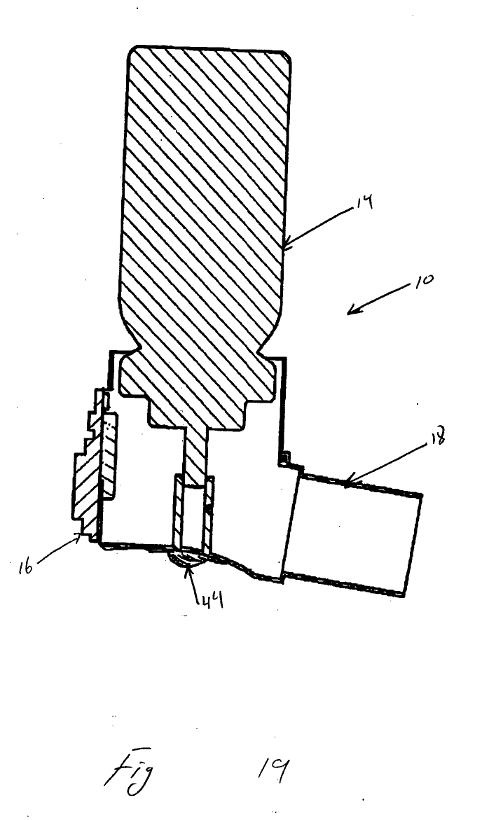

FIG. 17 shows a space saving arrangement wherein the circuit components are mounted in the bottom of the mouthpiece 18 utilizing flexible circuitry such as that used in cell phones. In the example shown in FIG. 18 the battery 30 is located in the bottom of the mouthpiece 18. Such an orientation minimizes the volume necessary for accommodation of the counter module 16. It should be understood that this embodiment may differ depending on whether the overall volume of the mouthpiece or the volume of air pathway is the critical concern.

FIG. 18 shows a dome switch 44 mounted directly in line with the actuator 28. When the canister 14 is moved down, the actuator 28 puts pressure on the switch and closes the circuit. The switch is electrically connected to the counter module 16.

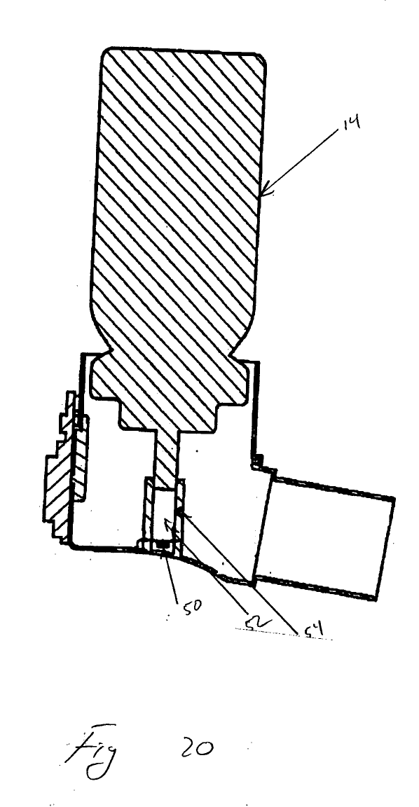

A further orientation of the elements of the present invention is shown in FIG. 19. In FIG. 19, a dome switch 44 is mounted externally on mouthpiece 18. The user holds the apparatus 10 by putting their thumb on the bottom of the mouthpiece 18 and squeezing the top of the canister 14 with their first two fingers. In the course of this squeezing action, the switch 44 on the bottom of the mouthpiece 18 is closed. In this case, the force required to close the switch must be carefully designed to provide accurate counting and prevent unintended closure of the switch 44.

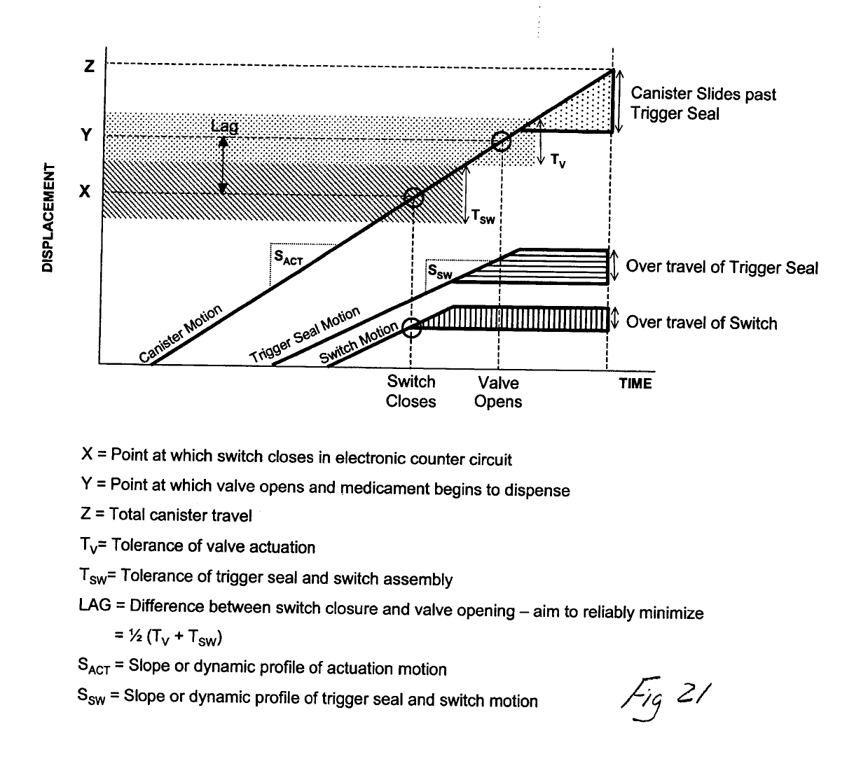

FIG. 20 shows a pressure sensor 50 that can detect the pressure in the actuator sump region 52. As the actuator 28 moves in the direction of the pressure sensor 50 the pressure in the sump will increase as the only point of egress for the air in the sump is through the orifice 54. Upon dispensation of the medication from the canister 14 the pressure in the actuator sump 52 is greatly increased. This increased pressure activates the pressure switch 50 and closes the electrical circuit. The pressure sensor 50 is electrically connected to the counter module 16.

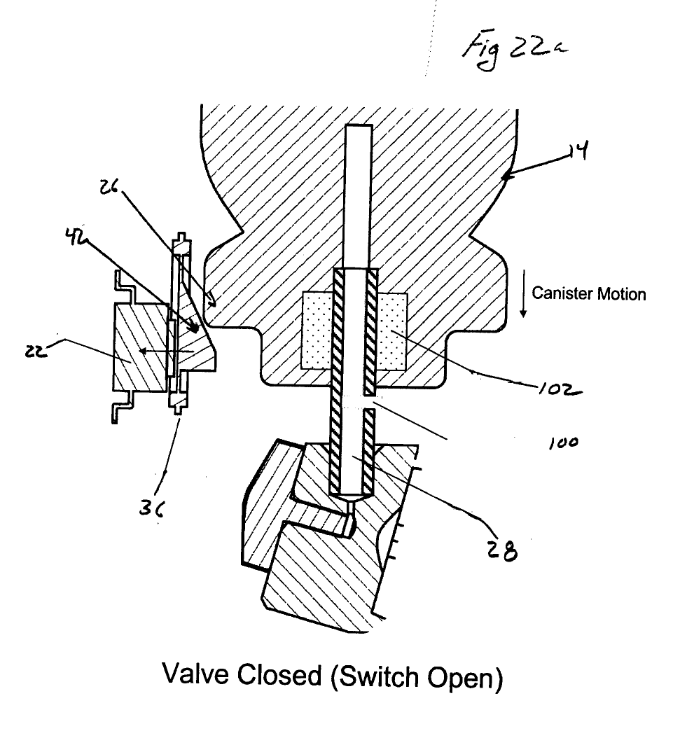

Another embodiment of the present invention is the optimization of switch-valve lag. The operation of an inhaler is shown graphically in FIG. 21. The displacement of the canister 14 in the holder 10 is charted along the Y-axis, and time is charted along the X-axis. In a perfect system, the switch on the counter would close at the instant the valve opens to dispense medication. Because all manufactured mechanical systems have dimensional variations (i.e. tolerances) associated with them, both the dispensing of the medicament from the canister 14 and the triggering of a count on the counter 16 will occur within a certain range of displacements (Tᵥ, T_{sw}). In order to assure maximum accuracy and reliability of the counter, the relative timing of these two events and their tolerances must be carefully managed. Specifically, it is paramount that under no circumstances will medicament be dispensed without triggering a count.

A practical consequence of the no missed count requirement and the tolerances associated with dispensing and counting is that the counter must be triggered immediately before the medicament is dispensed. This dictates that there is a lag in time and displacement between the nominal switch closure X and the nominal valve opening Y.

As shown in FIG. 21, the canister is moved a certain distance over a given time period. At some distance of travel, the canister contacts the trigger seal, this is indicated by the trigger seal motion line passing through the X axis. Shortly thereafter, the trigger seal contacts the switch, which in turn closes the contacts of the switch some time thereafter. In FIG. 21 closing of the contacts of the switch is represented by the dashed line titled “Switch Closes.” This is the first point, whether in time or distance, for the calculation of lag. As the canister continues to travel the switch remains closed. At some further distance of travel the valve opens, this is indicated in FIG. 21 by the notation “Valve Opens.” This represents the second point in time or distance for determination of lag. The difference in time or travel of the canister between the circuit closing and valve opening is the lag. Lag is shown in FIG. 21 as the distance between lines X and Y. Lag, in terms of time, is represented by the distance between the dashed lines titled “Switch Closes” and “Valve Opens.” Other features shown in FIG. 21 include tolerance ranges for both the switch and the valve, T_{sw} and Tᵥ, respectively. Another feature shown in FIG. 21 is over travel of the switch and the seal. The allowance for the over travel of these components accommodates the operational parameters of the valve without requiring tight tolerances.

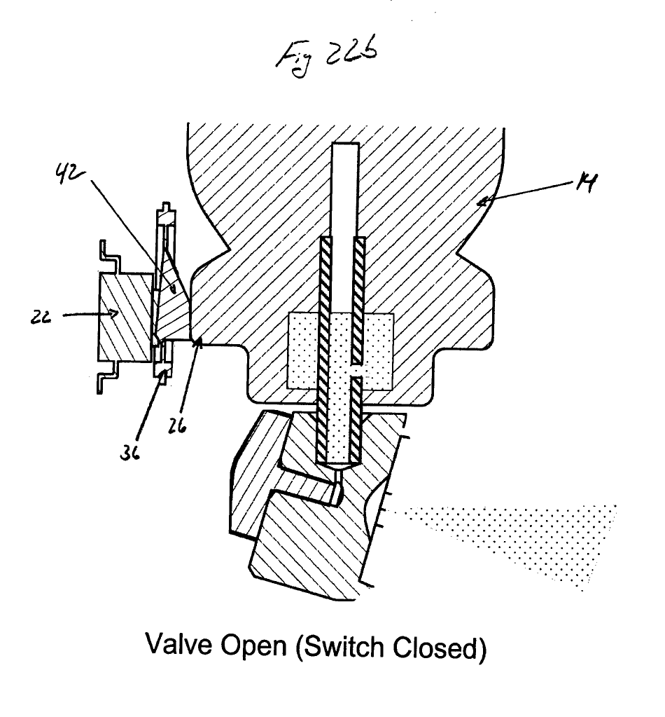

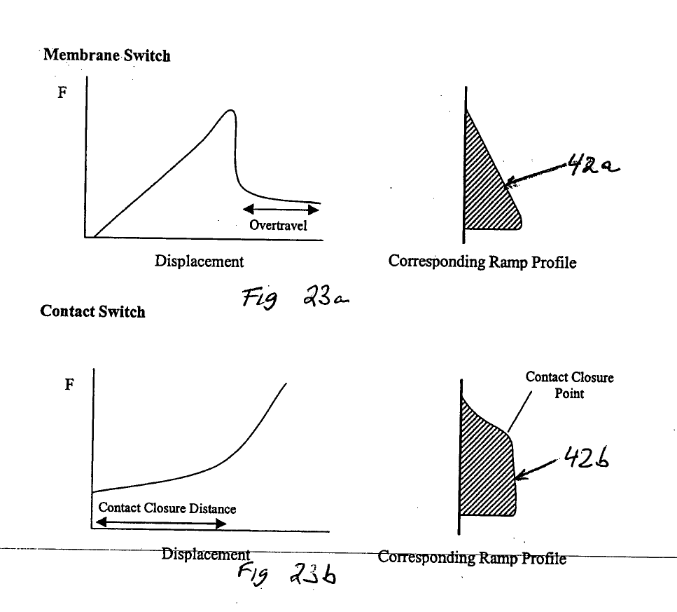

As described above with respect to FIG. 8, one aspect of the present invention is the use of an elastomeric seal 36 and ramp 42. This is shown in greater detail in FIGS. 22a and b, where it can be seen that as the canister 14 travels, the elastomeric or trigger seal 36 is displaced by the ferrule 26 thereby closing switch 22. As can be further seen from FIGS. 22a and b, the shape of the ramp 42 effects how the ferrule acts upon the elastomeric seal. By altering the shape of the ramp 42, the timing of the closing of the contacts can be altered to optimize the lag in the device. In any event, however, the switch closure must occur before the valve opening to prevent the scenario of dispensing medicine without registering a count.

As shown in FIG. 22a, the valve 100 is opened when the canister 14 is depressed, lowering the canister 14 relative to the stationary actuator 28. When the valve 100 accesses the drug reservoir chamber 102 the pressurized dose is expelled. The valve 100 has an inherent tolerance (T_{V}) (shown in FIG. 21) associated with insuring reliability in dispensing medicine at a known travel distance. Therefore in order to minimize counter lag it is necessary to minimize the tolerance associated with the switch assembly (T_{SW}). This is accomplished by adjusting the ramp design and material properties of the elastomeric seal.

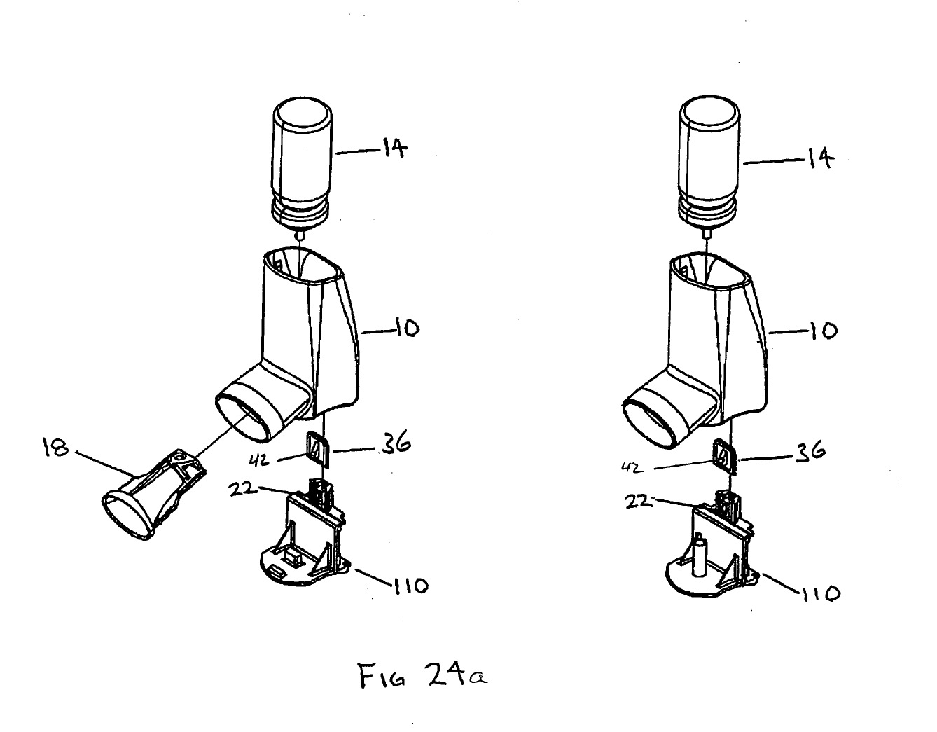

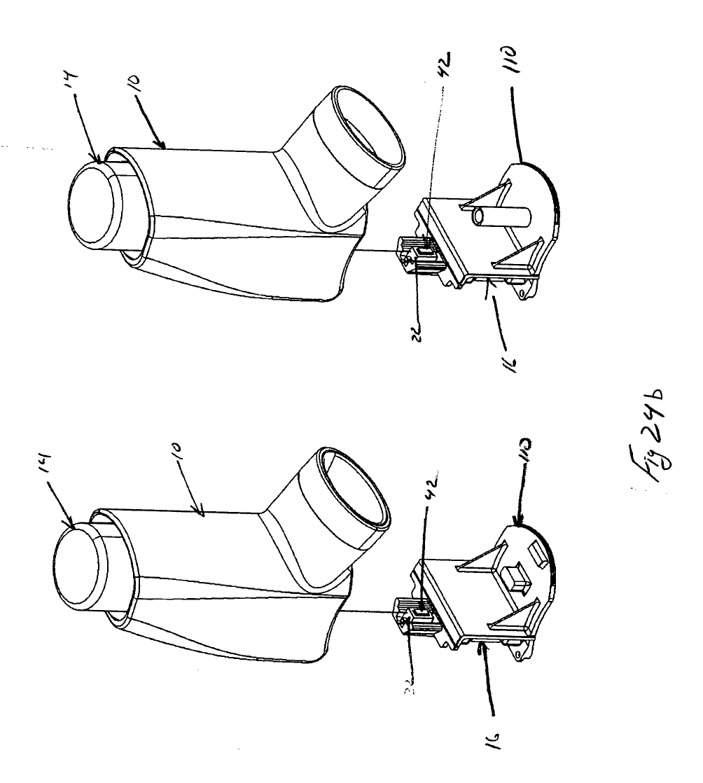

Design of the ramp allows precise control of switch dynamics (S_{SW}). Two such ramp designs are shown if FIGS. 23a and 23b. 23a shows one ramp design for a membrane switch. Similarly, 23b shows a ramp design for a contact switch. FIGS. 23a and b demonstrate that by changing the shape of the ramp 42a and b, the interaction between the switch force (F) and the displacement can be altered.

The use of the elastomeric membrane 36 and ramp 42 as a triggering device for the switch 22 has several distinct advantages. Initially the distance the canister must travel to close the switch can be easily changed without changing the switch or canister. Secondly, many conventional aerosol-metering valves operate based on motion of the valve stem with respect to the valve ferrule. In the present invention, the ramp acts directly on the valve ferrule, assuring the most accurate mechanical indication of valve opening. Further the ramp profile can be varied to complement switch force/displacement curve, and to accommodate timing and travel parameters of different metering valves or canister types. Still further, the properties of the elastomeric materials used in making the ramp (i.e., durometer, surface coefficient of friction) can be varied to accommodate valve and/or switch characteristics. For example, use of lower durometer (“softer”) material to allow ramp to “crush” when switch “bottoms out.” This allows for larger design tolerances. Other advantages to the use of elastomeric seals and ramps include a reduction in the number of parts for the inhaler. The elastomeric seal and ramp can use a return spring in the switch to return the ramp to its rest position after deflecting during valve actuation. Further, in a single component both seal and triggering components are combined.

Furthermore, this seal can be molded directly either into the actuator body 10 or onto the base housing 110 to form a two-piece assembly as shown in FIG. 24. This provides a low cost, “One Size Fits All” assembly and triggering solution. FIG. 24 shows the base housing 110 being used with two different nozzle geometries. In these configurations, the same electronic counter module 16 can be used with a variety of canisters and valves using different ramp geometries. This facilitates tighter tolerances while still eliminating the aberrant counting problems such as dispensing medicine without registering a count or not counting upon dispensation of medicine.The canister 14 with ferrule 26 is installed into the stem opening in the spray nozzle. Therefore, the nozzle determines the location of canister 14 and ferrule 26. By incorporating the nozzle geometry into the same base housing 110 that houses the switch 22, tolerance stackup is minimized. Consequently, the lag between the counting and dispensing is also minimized. Furthermore, given that the counter and nozzle functions are located in one relatively small subassembly, this embodiment is easily adapted to almost any canister and valve combination, as well as to a wide variety of actuator body 10 styles and sizes.

The two-piece design as shown in FIG. 24 allows different nozzle geometries to be molded into the base housing 110 while still utilizing the same actuator body 10 and counter module 16. This feature additionally accommodates various canisters as well as providing for relatively easy modification of nozzle performance through the use of different ramps.

Further, in production, only one injection mold tool need be used to produce a variety of ramp geometries. This is effectuated by simply changing inserts in the tool to form different ramps.

Accordingly, the use of elastomeric seals and ramps greatly increases the flexibility of the use of the dosage counter, and in particular a base housing 110 with a wide variety of inhaler, canister, switch, and nozzle varieties.

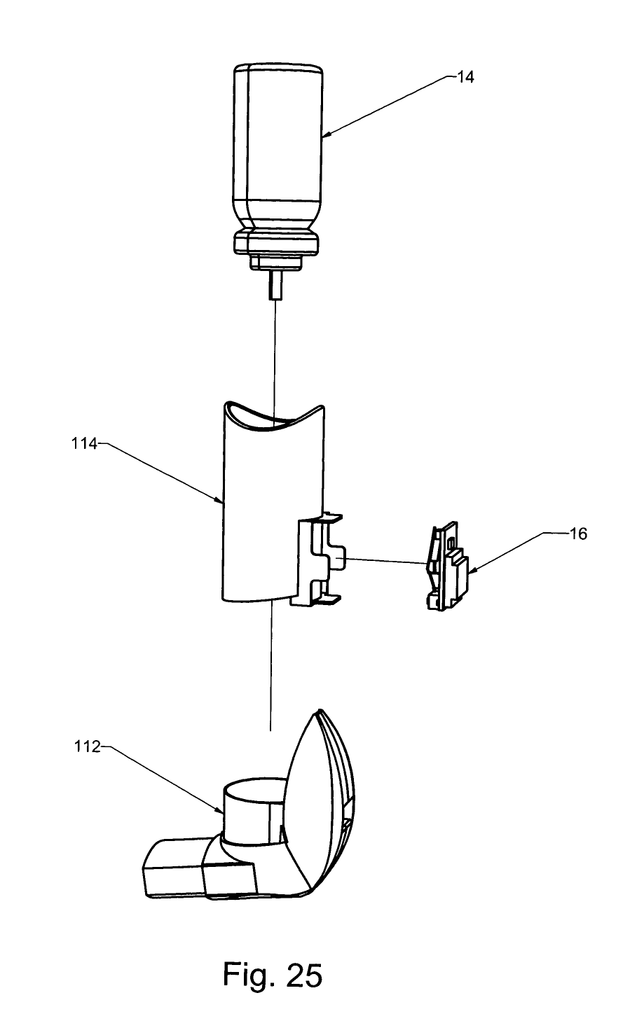

An alternate multi component design is shown in FIG. 25. The design allows the counter to be readily incorporated into an inhaler, which is comprised of two or more components, possibly of different materials and/or colors. The counter function is contained in one of the components, and nests within the other component. In FIG. 25, the counter 16 is located on the upper housing 114, and fits within the lower housing 112 upon assembly. Such an arrangement enables the counter to be incorporated into a variety of inhaler designs without the need for additional components.

While the invention has been described in connection with what is considered to be the most practical and preferred embodiment, it should be understood that this invention is not limited to the disclosed embodiments, but on the contrary, is intended to cover various modifications and equivalent arrangements included within the spirit and scope of the appended claims.

1. An apparatus for dispensing medication comprising:
at least one canister containing the medication to be dispensed, said canister being movable in a first and a second direction;
a mouthpiece providing a point of dispensation for the medication from the canister to a user when the canister is moved in the first direction;
a switch means for completing an electrical circuit when said canister moves in said first direction and opening the electrical circuit when said canister moves in the second direction, wherein the switch means is oriented to enable operational connectivity with the canister or canister discharge;
a counter module for performing a count upon the closure of the electrical circuit and displaying a dispensation history of the medication in the at least one canister; and
a seal isolating the counter module from the mouthpiece and the canister to prevent contamination.

2. The apparatus as recited in claim 1, wherein the dispensation history includes the number of doses of medication remaining in the canister.

3. The apparatus as recited in claim 1, wherein the dispensation history includes the number of doses taken of a dosage sequence.

4. The apparatus as recited in claim 1, wherein the dispensation history includes the number of doses taken over a period of time.

5. The apparatus of claim 4, wherein the period of time can be varied by a user.

6. The apparatus of claim 1, wherein the dispensation history includes time since the last dispensation of the medication.

7. The apparatus as recited in claim 1, wherein a display portion of the counter module is on the front of the mouthpiece.

8. The apparatus as recited in claim 1, wherein a display portion of the counter module is on the back of the mouthpiece.

9. The apparatus as recited in claim 1, wherein the display portion of the counter module is on the side of the mouthpiece.

10. The apparatus of claim 1, wherein the switch means includes an electrically conductive contact imbedded in the seal.

11. The apparatus of claim 1, wherein at least portion of the counter module is disposed in the mouthpiece.

12. The apparatus of claim 1, wherein at least a portion of the counter module is disposed external to the mouthpiece.

13. The apparatus of claim 1, wherein the seal includes a ramp that acts upon the switch means when the canister is moved in the first direction.

14. The apparatus of claim 1, wherein the switch means is mounted on a circuit board and is acted upon by a ferrule portion of the canister, the switch means being isolated from the canister by a second seal.

15. The apparatus of claim 1, wherein the seal is made of conductive material.

16. The apparatus of claim 1, wherein the switch means is a water resistant dome switch.

17. The apparatus of claim 16, wherein the dome switch is mounted substantially parallel to an axis of travel of the canister and is acted upon by a ferrule of the canister.

18. The apparatus of claim 16, wherein the dome switch is mounted on a platform that extends perpendicular to the axis of travel of the canister into the mouthpiece and is acted upon by an end portion of the canister.

19. The apparatus of claim 16, wherein the dome switch is mounted on a top surface of an actuator sump and is acted upon by an end portion of the canister.

20. The apparatus of claim 16, wherein the switch means is comprised of:
at least two open contacts that are in electrical communication with the counter module; and
a conductive surface of the canister to close the contacts when the canister moves in the first direction.

21. The apparatus of claim 20, where in the at least two open contacts are located on a top surface of an actuator sump and are acted upon by a metallic end portion of the canister.

22. The apparatus of claim 16, wherein the dome switch is located an actuator sump and is acted upon by the actuator as the canister moves in the first direction.

23. The apparatus of claim 16, wherein the dome switch is located on an exterior surface of the mouthpiece and is depressed when the user depresses the canister against the mouthpiece to move the canister in the first direction.

24. The apparatus of claim 1, wherein the switch means is formed of a movement sensor.

25. The apparatus of claim 24, wherein the movement sensor is a light sensor, said light sensor emitting light and receiving a reflected signal, upon movement of the canister the reflected signal is altered and the altered signal is detected by the sensor providing input to the circuitry and changes the count.

26. The apparatus of claim 25, wherein the sensor is located to act upon and detect a changing position of the canister as it is moved in the first direction.

27. The apparatus of claim 24, wherein the movement sensor is an acoustic sensor, said acoustic sensor emitting an acoustic signal and receiving a reflected signal, upon movement of the canister the reflected signal is altered, and the altered signal is detected by the sensor closing contacts housed therein.

28. The apparatus of claim 24, wherein the movement sensor is an acoustic sensor, said acoustic sensor senses the acoustic signature of the aersolization of a metered dose dispensation.

29. The apparatus of claim 27, wherein the sensor is located to act upon and detect a changing position of the canister as it is moved in the first direction.

30. The apparatus of claim 24, wherein the movement sensor is a magnet sensor, upon movement of the canister the magnetic signature of the canister is altered which is detected by the sensor providing input to the circuitry and changes the count.

31. The apparatus of claim 30, wherein the sensor is located to act upon and detect a changing geometry of the canister as it is moved in the first direction.

32. The apparatus of claim 24, wherein the sensor is a pressure sensor.

33. The apparatus of claim 32, wherein the pressure sensor is located in an actuator sump and detects a change in pressure upon the dispensation of the medication from the canister.

34. An apparatus for dispensing medication comprising:
at least one canister containing the medication to be dispensed, said canister being movable in a first and a second direction;
a switch means for completing an electrical circuit when said canister moves in said first direction and opening the electrical circuit when said canister moves in the second direction;
a counter module for performing a count upon the closure of the electrical circuit and displaying a dispensation history of the medication in the at least one canister; and
a ramp seal isolating the switch means from the canister to prevent contamination, wherein the ramp seal is acted upon by the canister upon movement of the canister in the first direction and wherein the ramp seal acts on the switch means to close the electrical circuit.

35. The apparatus as recited in claim 34, wherein the dispensation history includes the number of doses of medication remaining in the canister.

36. The apparatus as recited in claim 34, wherein the dispensation history includes the number of doses taken of a dosage sequence.

37. The apparatus as recited in claim 34, wherein the dispensation history includes the number of doses taken over a period of time.

38. The apparatus of claim 37, wherein the period of time can be varied by a user.

39. The apparatus of claim 34, wherein the dispensation history includes time since the last dispensation of the medication.

40. The apparatus as recited in claim 34, wherein a display portion of the counter module is on the front of the mouthpiece.

41. The apparatus as recited in claim 34, wherein a display portion of the counter module is on the back of the mouthpiece.

42. The apparatus as recited in claim 34, wherein the display portion of the counter module is on the side of the mouthpiece.

43. The apparatus of claim 34, wherein the switch means includes an electrically conductive contact imbedded in the seal.

44. The apparatus of claim 34 wherein a ferrule portion of the canister acts upon the ramp seal.

45. The apparatus of claim 34, wherein the counter, and ramp seal are formed in a common component.

46. The apparatus of claim 34 further comprising a sump for a nozzle of the canister wherein said counter, ramp seal and sump are formed as a common component.

47. The apparatus as recited in claim 45 wherein the common component is injection moldable.

48. The apparatus as recited in claim 46 wherein the common component is injection moldable.

49. The apparatus as recited in claim 45 wherein the common component is adaptable to canister holders for a variety of canister shapes and sizes.

50. The apparatus as recited in claim 46 wherein the common component is adaptable to canister holders for a variety canister of shapes and sizes.

51. The apparatus of claim 34 wherein the counter is adaptable to canister holders for a variety of canister shapes and sizes.

52. The apparatus of claim 34 formed of components including a canister holder, a canister, a mouthpiece, and a counter, wherein the counter is adaptable for use with a canister holder, canister, and a mouthpiece of a variety of sizes and shapes.

53. The apparatus of claim 1 wherein the counter module is adaptable to canister holders for a variety of canister shapes and sizes.

54. The apparatus of claim 1 formed of components including a canister holder, a canister, a mouthpiece, and a counter, wherein the counter is adaptable for use with a canister holder, canister, and a mouthpiece of a variety of sizes and shapes.
